(19) United States
(12) Patent Application Publication
(10) Pub. No.: US 2020/0087360 A1
(43) Pub. Date: Mar. 19, 2020

(54) Method of treating a diarrhea disorder using a novel polypeptide

(71) Applicant: The Regents of the University of California, Oakland, CA (US)

(72) Inventors: Yiwen Zhu, Albany, CA (US); Len A. Pennacchio, Sebastopol, CA (US)

(73) Assignee: The Regents of the University of California, Oakland, CA (US)

(21) Appl. No.: 16/419,860

(22) Filed: May 22, 2019

Related U.S. Application Data
(60) Provisional application No. 62/675,099, filed on May 22, 2018.

(51) Int. Cl. C07K 14/435; C12N 15/86

(52) U.S. Cl. CPC C07K 14/435; A61K 38/00; C12N 2740/15043; C12N 15/86

(57) ABSTRACT

The present invention provides for a recombinant or isolated polypeptide comprising the amino acid sequence of an enhancer polypeptide associated with a diarrhea disorder; a transgenic non-human mammal, wherein the mammal is deleted or knocked out for one or more of an intestine-critical region (ICR); a pharmaceutical composition comprising the polypeptide of the present invention and a pharmaceutical acceptable carrier; and, a method of treating or preventing a subject suffering or at risk or suspected of suffering from a diarrhea disease or disorder, the method comprising administrating a pharmaceutical composition of the present invention to a subject in need of such treatment.

### RELATED PATENT APPLICATIONS

This application claims priority to U.S. Provisional Patent Application Ser. No. 62/675,099, filed on May 22, 2018, which is hereby incorporated by reference.

### STATEMENT OF GOVERNMENTAL SUPPORT

The invention was made with government support under Contract No. DE-AC02-05CH11231 awarded by the U.S. Department of Energy and Grant No. HG003988 awarded by the National Institutes of Health. The government has certain rights in the invention.

### FIELD OF THE INVENTION

The present invention is in the field of methods of treating a diarrhea disorder.

### BACKGROUND OF THE INVENTION

Whole exome sequencing (WES) is a powerful approach for the identification of causal mutations of protein-coding sequences in rare human disorders¹. However, this approach generally fails to interrogate the remaining non-coding 98% of the human genome, despite strong emerging indications that a significant proportion of disease-associated variants affect non-coding functions^{2,3}. While whole genome sequencing (WGS) is increasingly utilized and can in principle identify both coding and non-coding mutations, it raises the significant difficulty of interpreting non-coding sequence changes for functional relevance. This is a particular challenge for regulatory sequences located distant from known protein-coding genes because the exact positions and in vivo functions of most such distant-acting regulatory sequences in the human genome remain poorly annotated. Furthermore, the in vivo consequences of changes to these sequences are considerably more difficult to predict than those in protein-coding sequences. In contrast to coding mutations, a very limited number of sequence changes affecting human distant-acting regulatory elements associated with severe phenotypes have been identified, and even fewer are understood at the mechanistic level⁴.

### SUMMARY OF THE INVENTION

The present invention provides for a recombinant or isolated polypeptide comprising the amino acid sequence of an enhancer polypeptide.

In some embodiments, the amino acid sequence comprises at least 70% identity of SEQ ID NO:1, SEQ ID NO:2, or SEQ ID NO:3.

The amino acid sequence of the mouse enhancer polypeptide is as follows:

(SEQ ID NO: 1)
MAAGVIRSVC DFRLPLPSHE SFLPIDLEAP EISEEEEEEE
EEEEEEEEEE EVDQDQQGEG SQGCGPDSQS SGVVPQDPSS
PETPMQLLRF SELISGDIQR YFGRKDTGQD PDAQDIYADS
QPASCSARDL YYADLVCLAQ DGPPEDEEAA EFRMHLPGGP
EGQVHRLGHR GDRVPPLGPL AELFDYGLRQ FSRPRISACR
RLRLERKYSH ITPMTQRKLP PSFWKEPVPN PLGLLHVGTP
DFSDLLASWS AEGGSELQSG GTQGLEGTQL AE

The amino acid sequence of the human enhancer polypeptide is as follows:

(SEQ ID NO: 2)
MAAGVIRPLC DFQLPLLRHH PFLPSDPEPP ETSEEEEEEE
EEEEEEEGEG EGLGGCGRIL PSSGRAEATE EAAPEGPGSP
ETPLQLLRFS ELISDDIRRY FGRKDKGQDP DACDVYADSR
PPRSTARELY YADLVRLARG GSLEDEDTPE PRVPQGQVCR
PGLSGDRAQP LGPLAELFDY GLQQYWGSRA AAGWSLTLER
KYGHITPMAQ RKLPPSFWKE PTPSPLGLLH PGTPDFSDLL
ASWSTEACPE LPGRGTPALE GARPAE

The amino acid sequence of the longer mouse enhancer polypeptide is as follows:

(SEQ ID NO: 3)
MHVEPLLHPS ACVCCSREPQ NFGDLNK
MAAGVIRSVC DFRLPLPSHE SFLPIDLEAP EISEEEEEEE
EEEEEEEEEE EVDQDQQGEG SQGCGPDSQS SGVVPQDPSS
PETPMQLLRF SELISGDIQR YFGRKDTGQD PDAQDIYADS
QPASCSARDL YYADLVCLAQ DGPPEDEEAA EFRMHLPGGP
EGQVHRLGHR GDRVPPLGPL AELFDYGLRQ FSRPRISACR
RLRLERKYSH ITPMTQRKLP PSFWKEPVPN PLGLLHVGTP
DFSDLLASWS AEGGSELQSG GTQGLEGTQL AEV

In some embodiments, the polypeptide comprises one or more of the following amino acid sequences: MAAGVIR (SEQ ID NO: 4), SEEEEEEEEEEEEEE (SEQ ID NO: 5), SPETP (SEQ ID NO: 6), QLLRFSELIS (SEQ ID NO: 7), RYFGRKD (SEQ ID NO: 8), GQDPDA (SEQ ID NO: 9), LYYADLV (SEQ ID NO: 10), PLGPLAELFDYGL (SEQ ID NO: 11), LERKY (SEQ ID NO: 12), HITPM (SEQ ID NO: 13), QRKLPPSFWKEP (SEQ ID NO: 14), PLGLLH (SEQ ID NO: 15), and GTPDFSDLLASWS (SEQ ID NO: 16). In some embodiments, the polypeptide comprises two or more, three or more, four or more, five or more, six or more, seven or more, eight or more, nine or more, ten or more, eleven or more, or twelve or more of amino acid sequences SEQ ID NOs: 4-16. In some embodiments, the polypeptide comprises one or more, two or more, three or more, four or more, five or more, six or more, seven or more, eight or more, nine or more, ten or more, eleven or more, or twelve or more, or all, of the individual and/or consecutive stretches of amino acid residues that are identical between the two sequences indicated with an asterisks (“*”) in FIG. 13.

In some embodiments, the amino acid sequence comprises at least 80%, 90%, 95%, or 99% identity of SEQ ID NO:1, SEQ ID NO:2, or SEQ ID NO:3.

The present invention also provides for a nucleic acid encoding the polypeptide of the present invention.

The present invention also provides for a host cell comprising the nucleic acid encoding the polypeptide of the present invention capable of expressing the polypeptide.

The present invention also provides for a method for synthesizing and/or purification/isolation of the polypeptide and/or nucleic acid of the present invention.

The present invention also provides for a transgenic non-human mammal, wherein the mammal is deleted or knocked out for one or more of an intestine-critical region (ICR). In some embodiments, the mammal is a mouse or rat.

The present invention also provides for a pharmaceutical composition comprising the polypeptide of the present invention and a pharmaceutically acceptable carrier.

The present invention also provides for a method of treating or preventing a subject suffering or at risk or suspected of suffering from a diarrhea disease or disorder, the method comprising administrating a pharmaceutical composition of the present invention to a subject in need of such treatment.

In some embodiments, the subject is a mammal. In some embodiments, the mammal is human. In some embodiments, the subject is suffering from a diarrhea disease or disorder. In some embodiments, the subject at risk or suspected of suffering from a diarrhea disease or disorder. In some embodiments, the diarrhea disease or disorder is a congenital diarrhea disorder, or a severe congenital malabsorptive diarrhea.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1A. Overview of human and mouse locus and key findings. Family pedigrees and genotyping results for patients compound heterozygous for the two deletion alleles.

FIG. 1B. Overview of human and mouse locus and key findings. Family pedigrees and genotyping results for patient homozygous for one of the deletion alleles.

FIG. 1C. Overview of human and mouse locus and key findings. Patient 4.2 at birth and at age 2y with total parenteral nutrition (TPN).

FIG. 1D. Overview of human and mouse locus and key findings. Genomic map of the deletion alleles in human, indicating the location of ΔL and ΔS, as well as their minimal overlapping region ICR. Exome sequencing data is capped at up to 5 overlapping tags; vertebrate conservation is 100-vertebrate PhyloP; only selected transcription factor binding sites and DHS clusters with signal in >20/125 ENCODE cell types shown.

FIG. 1E. Overview of human and mouse locus and key findings. Genomic map of the deletion alleles in mouse, indicating the location of ΔL and ΔS, as well as their minimal overlapping region ICR. Exome sequencing data is capped at up to 5 overlapping tags; vertebrate conservation is 100-vertebrate PhyloP; only selected transcription factor binding sites and DHS clusters with signal in >20/125 ENCODE cell types shown.

FIG. 1F. Overview of human and mouse locus and key findings. General appearance of wildtype and chr17^{ΔICR/ΔICR} mice at 21 days after birth, showing overall significantly reduced size.

FIG. 1G. Overview of human and mouse locus and key findings. Abnormal appearance of fecal pellets from chr17^{ΔICR/ΔICR} mice.

FIG. 2A. Enhancer activity of the ICR and mouse deletion phenotypes. Enhancer reporter activity in E13.5 and E14.5 transgenic mouse embryos. Cross-sections showing X-gal staining for β-galactosidase activity in E13.5 stomach, pancreas and duodenum as marked.

FIG. 2B. Enhancer activity of the ICR and mouse deletion phenotypes. Enhancer reporter activity in E13.5 and E14.5 transgenic mouse embryos. E14.5 cross-section showing immunofluorescence with anti-β-galactosidase (ICR enhancer activity, red), anti-endomucin (endothelial cells, green), and DAPI (DNA, blue).

FIG. 2C. Enhancer activity of the ICR and mouse deletion phenotypes. Enhancer reporter activity in E13.5 and E14.5 transgenic mouse embryos. Chr17^{ΔICR/ΔICR} offspring are viable but show a reduction in size and weight compared to wild-type littermates.

FIG. 2D. Enhancer activity of the ICR and mouse deletion phenotypes. Reduction in body weight among surviving offspring of chr17^{ΔICR/ΔICR} compared to wild-type. Body weight of female mice shown here; male wildtype and chr17^{ΔICR/ΔICR} mice had higher mean weights with similar genotype-dependent weight differences.

FIG. 2E. Enhancer activity of the ICR and mouse deletion phenotypes.

Increased mortality of chr17^{ΔICR/ΔICR} compared to wild-type.

FIG. 3A: Human enteroendocrine cell development is impaired in iPSC-derived intestinal organoid cultures. Human intestinal organoids (HIOs) are generated from control (+/+), carrier (+/ΔL), and patient (ΔL/ΔL) iPSC lines and analyzed at 21 days and 42 days of culture. Intestinal epithelial development is interrogated by expression of the epithelial markers FOXA2 (blue) and CDH1 (red). Synaptophysin (SYP—green) is used to mark developing enteroendocrine cells. Representative examples from two separate iPSC lines from each patient run in triplicate are shown.

FIG. 3B: Human enteroendocrine cell development is impaired in iPSC-derived intestinal organoid cultures. Analysis of 42 day HIOs by quantitative RT-PCR for the enteroendocrine markers ARX, Chromogranin A (CHGA) and synaptophysin (SYP). Error bars show standard error of the mean. Control vs. carrier is not significant. Carrier vs patient is significant at p<0.05 in all cases (student's t-test, one-tailed). Results are from two separate iPSC lines from each patient run in triplicate.

FIG. 4. Family pedigrees. Filled black symbols are affected, and deletion genotypes are indicated in red. Exome sequencing is done for individuals 1.1, 2.1, 3.1, 4.1, 4.2; whole genome sequencing is done for individual 2.1. Transcriptome analysis done for 2.1, 2.4. Patient 1.1 (*) is found to have uniparental disomy (UPD).

FIG. 5: Whole genome linkage analysis. Analysis of SNP genotyping is performed on six of the patients in families 1-5 and their 22 relatives detected a single significant telomeric linkage interval on chr16 with a max LODscore of 4.26. Haplotype reconstruction confirm this interval with flanking marker rs207435 (chr16: 2,984,868) and show two distinct disease haplotypes in an either homozygous setting in affected individuals for disease allele 1 (i.e. ΔL) in families 2, 3, 5, or a compound heterozygous setting for disease alleles 1 and 2 (i.e. ΔS) in family 4. All affected individuals carrying disease allele 1 show an identical disease haplotype from rs533184 (chr16: 1,155,025) to rs397435 (chr16: 2,010,138). The affected girl in family 1 show uniparental disomy for disease allele 1, i.e. maternal isodisomy, within this interval.

FIG. 6: Schematic of reads covering exons in the C16orf91 gene, for the five exome-sequenced patients and for three controls sequenced under identical conditions. The first three patients with a ⊗L/⊗L genotype have zero-coverage in the three upstream exons (right). The last two patients with a ⊗L/⊗S genotype have non-zero coverage in these exons, but significantly lower than controls. The downstream exons (left) have high coverage in all subjects. Numbers indicate scale in sequencing reads per base.

FIG. 7A: Targeted deletion of the ICR non-coding sequence in mice. Overview of targeting approach. See Methods for details.

FIG. 7B: Targeted deletion of the ICR non-coding sequence in mice. Genotyping results obtained from genomic DNA isolated from the tails of homozygous and heterozygous ICR deletion mice, compared to a wild type control. See Methods for primers and details.

FIG. 8. Modified intestinal content in the wild-type (left) and the chr17^{ΔICR/ΔICR} mouse (right).

FIG. 9A. IRS deletion causes changes in intestinal and fecal microbiome composition. Microbial communities in different intestinal compartments and feces are profiled by 16S rRNA-based sequence profiling. Family-level relative abundance profiles of the top fifteen most abundant prokaryotic families for wildtype and chr17^{ΔICR/ΔICR} intestinal and fecal samples, organized by sample type. The most pronounced changes are observed in colon and fecal samples.

FIG. 9B. IRS deletion causes changes in intestinal and fecal microbiome composition. Microbial communities in different intestinal compartments and feces are profiled by 16S rRNA-based sequence profiling. Family-level relative abundance profiles of the top fifteen most abundant prokaryotic families for wildtype and chr17^{ΔICR/ΔICR} intestinal and fecal samples, organized by sample type. The most pronounced changes are observed in colon and fecal samples.

FIG. 9C. IRS deletion causes changes in intestinal and fecal microbiome composition. Microbial communities in different intestinal compartments and feces are profiled by 16S rRNA-based sequence profiling. Box plots of Shannon's diversity for all fecal samples group into wildtype and chr17^{ΔICR/ΔICR} sample types.

FIG. 10. Increased immunoreactivity of Chromogranin A stained enteroendocrine cells in duodenal biopsy (villi and intestinal glands) of patient 7.1 (A) as compared with the number in a control sample (C), and in the antral glands of stomach (pyloric mucosae) biopsy of patient 2.1 (B) as compared with the number in a control sample (D).

FIG. 11. HIOs generated from affected patient, carrier and wild-type control all showing normal morphology.

FIG. 12. Affected patient, carrier and wild-type control-iPSC line's showing normal karyotype.

FIG. 13. Comparison of amino acid sequences between SEQ ID NO: 1 and SEQ ID NO:2. Amino acid residues that are identical between the two sequences are indicated with an asterisks (“*”).

### DETAILED DESCRIPTION OF THE INVENTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

As used in the specification and the appended claims, the singular forms “a”, “an”, and “the” include plural references unless the context clearly dictates otherwise. Thus, for example, reference to a “polypeptide” includes a single polysaccharide molecule, and a plurality of polysaccharide molecules having the same, or similar, chemical formula, chemical and/or physical properties.

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

These and other objects, advantages, and features of the invention will become apparent to those persons skilled in the art upon reading the details of the invention as more fully described below.

### REFERENCES CITED

1 Bamshad, M. J. et al. Exome sequencing as a tool for Mendelian disease gene discovery. Nature reviews. Genetics 12, 745-755, doi:10.1038/nrg3031 (2011).
2 Manolio, T. A. et al. Finding the missing heritability of complex diseases. Nature 461, 747-753, doi: 10.1038/nature08494 (2009).
3 Visel, A., Rubin, E. M. & Pennacchio, L. A. Genomic views of distant-acting enhancers. Nature 461, 199-205, doi: 10.1038/nature08451 (2009).
4 Dickel, D. E., Visel, A. & Pennacchio, L. A. Functional anatomy of distant-acting mammalian enhancers. Philosophical transactions of the Royal Society of London. Series B, Biological sciences 368, 20120359, doi:10.1098/rstb.2012.0359 (2013).
5 Avery, G. B., Villavicencio, O., Lilly, J. R. & Randolph, J. G. Intractable diarrhea in early infancy. Pediatrics 41, 712-722 (1968).
6 Straussberg, R. et al. Congenital intractable diarrhea of infancy in Iraqi Jews. Clinical genetics 51, 98-101 (1997).
7 Canani, R. B. & Terrin, G. Recent progress in congenital diarrheal disorders. Current gastroenterology reports 13, 257-264, doi:10.1007/s11894-011-0188-6 (2011).
8 Breil, T., Longerich, T., Bettendorf, M., Schnitzler, P. & Engelmann, G. An unusual intestinal infection causing intractable diarrhoea of infancy. Journal of clinical virology: the official publication of the Pan American Society for Clinical Virology 50, 97-99, doi: 10.1016/j.jcv.2010.10.012 (2011).
9 Qu, H. & Fang, X. A brief review on the Human Encyclopedia of DNA Elements (ENCODE) project. Genomics, proteomics & bioinformatics 11, 135-141, doi:10.1016/j.gpb.2013.05.001 (2013).
10 Calo, E. & Wysocka, J. Modification of enhancer chromatin: what, how, and why? Mol Cell 49, 825-837, doi: 10.1016/j.molcel.2013.01.038 (2013).
11 Eeckhoute, J. et al. Cell-type selective chromatin remodeling defines the active subset of FOXA1-bound enhancers. Genome Res 19, 372-380, doi: 10.1101/gr.084582.108 (2009).
12 Pennacchio, L. A. et al. In vivo enhancer analysis of human conserved non-coding sequences. Nature 444, 499-502, doi:10.1038/nature05295 (2006).
13 Gunawardene, A. R., Corfe, B. M. & Staton, C. A. Classification and functions of enteroendocrine cells of the lower gastrointestinal tract. International journal of experimental pathology 92, 219-231, doi:10.1111/j.1365-2613.2011.00767.x (2011).
14 Helander, H. F. & Fandriks, L. The enteroendocrine “letter cells”—time for a new nomenclature? Scandinavian journal of gastroenterology 47, 3-12, doi: 10.3109/00365521.2011.638391 (2012).
15 Yang, J., Brown, M. S., Liang, G., Grishin, N. V. & Goldstein, J. L. Identification of the acyltransferase that octanoylates ghrelin, an appetite-stimulating peptide hormone. Cell 132, 387-396, doi: 10.1016/j.cell.2008.01.017 (2008).
16 Gahete, M. D. et al. Metabolic regulation of ghrelin O-acyl transferase (GOAT) expression in the mouse hypothalamus, pituitary, and stomach. Molecular and cellular endocrinology 317, 154-160, doi: 10.1016/j.mce.2009.12.023 (2010).
17 Beucher, A. et al. The homeodomain-containing transcription factors Arx and Pax4 control enteroendocrine subtype specification in mice. PloS one 7, e36449, doi:10.1371/journal.pone.0036449 (2012).
18 Gecz, J., Cloosterman, D. & Partington, M. ARX: a gene for all seasons. Current opinion in genetics & development 16, 308-316, doi: 10.1016/j.gde.2006.04.003 (2006).
19 Itoh, M. et al. Partial loss of pancreas endocrine and exocrine cells of human ARX-null mutation: consideration of pancreas differentiation. Differentiation; research in biological diversity 80, 118-122, doi: 10.1016/j.diff.2010.05.003 (2010).
20 Du, A. et al. Arx is required for normal enteroendocrine cell development in mice and humans. Dev Biol 365, 175-188, doi:10.1016/j.ydbio.2012.02.024 (2012).
21 Kim, O. et al. GKN2 contributes to the homeostasis of gastric mucosa by inhibiting GKN1 activity. Journal of cellular physiology 229, 762-771, doi: 10.1002/jcp.24496 (2014).
22 Laurell, T. et al. A novel 13 base pair insertion in the sonic hedgehog ZRS limb enhancer (ZRS/LMBR1) causes preaxial polydactyly with triphalangeal thumb. Human mutation 33, 1063-1066, doi: 10.1002/humu.22097 (2012).
23 Kasowski, M. et al. Extensive variation in chromatin states across humans. Science 342, 750-752, doi: 10.1126/science. 1242510 (2013).
24 Ghiasvand, N. M. et al. Deletion of a remote enhancer near ATOH7 disrupts retinal neurogenesis, causing NCRNA disease. Nat Neurosci 14, 578-586, doi: 10.1038/nn.2798 (2011).
25 D'Haene, B. et al. Disease-causing 7.4 kb cis-regulatory deletion disrupting conserved non-coding sequences and their interaction with the FOXL2 promotor: implications for mutation screening. PLoS genetics 5, e1000522, doi: 10.1371/journal.pgen. 1000522 (2009).
26 Emison, E. S. et al. A common sex-dependent mutation in a RET enhancer underlies Hirschsprung disease risk. Nature 434, 857-863, doi:10.1038/nature03467 (2005).
27 Mellitzer, G. et al. Loss of enteroendocrine cells in mice alters lipid absorption and glucose homeostasis and impairs postnatal survival. The Journal of clinical investigation 120, 1708-1721, doi: 10.1172/JCI40794 (2010).
28 Mali, P., Esvelt, K. M. & Church, G. M. Cas9 as a versatile tool for engineering biology. Nature methods 10, 957-963, doi: 10.1038/nmeth.2649 (2013).
29 Li, H. & Durbin, R. Fast and accurate short read alignment with Burrows-Wheeler transform. Bioinformatics 25, 1754-1760, doi: 10.1093/bioinformatics/btp324 (2009).
30 Li, H. et al. The Sequence Alignment/Map format and SAMtools. Bioinformatics 25, 2078-2079, doi: 10.1093/bioinformatics/btp352 (2009).
31 Ge, D. et al. SVA: software for annotating and visualizing sequenced human genomes. Bioinformatics 27, 1998-2000, doi: 10.1093/bioinformatics/btr317 (2011).
32 Zhu, M. et al. Using ERDS to infer copy-number variants in high-coverage genomes. American journal of human genetics 91, 408-421, doi: 10.1016/j.ajhg.2012.07.004 (2012).
33 Trapnell, C. et al. Differential gene and transcript expression analysis of RNA-seq experiments with TopHat and Cufflinks. Nature protocols 7, 562-578, doi:10.1038/nprot.2012.016 (2012).
34 Bockenhauer, D. et al. Epilepsy, ataxia, sensorineural deafness, tubulopathy, and KCNJ10 mutations. The New England journal of medicine 360, 1960-1970, doi: 10.1056/NEJMoa0810276 (2009).
35 Purcell, S. et al. PLINK: a tool set for whole-genome association and population-based linkage analyses. American journal of human genetics 81, 559-575, doi: 10.1086/519795 (2007).
36 Lindemann, S. R. et al. The epsomitic phototrophic microbial mat of Hot Lake, Washington: community structural responses to seasonal cycling. Frontiers in microbiology 4, 323, doi:10.3389/fmicb.2013.00323 (2013).
37 Kunisato, A. et al. Direct generation of induced pluripotent stem cells from human nonmobilized blood. Stem cells and development 20, 159-168, doi:10.1089/scd.2010.0063 (2011).
38 Warlich, E. et al. Lentiviral vector design and imaging approaches to visualize the early stages of cellular reprogramming. Molecular therapy: the journal of the American Society of Gene Therapy 19, 782-789, doi:10.1038/mt.2010.314 (2011).
39 Spence, J. R. et al. Directed differentiation of human pluripotent stem cells into intestinal tissue in vitro. Nature 470, 105-109, doi: 10.1038/nature09691 (2011).
40 McCracken, K. W., Howell, J. C., Wells, J. M. & Spence, J. R. Generating human intestinal tissue from pluripotent stem cells in vitro. Nature protocols 6, 1920-1928, doi:10.1038/nprot.2011.410 (2011).
41 Takahashi, K. et al. Induction of pluripotent stem cells from adult human fibroblasts by defined factors. Cell 131, 861-872, doi: 10.1016/j.cell.2007.11.019 (2007).
42 Glusman, G., Caballero, J., Mauldin, D. E., Hood, L. & Roach, J. C. Kaviar: an accessible system for testing SNV novelty. Bioinformatics 27, 3216-3217, doi: 10.1093/bioinformatics/btr540 (2011).
43 Abecasis, G. R. et al. A map of human genome variation from population-scale sequencing. Nature 467, 1061-1073, doi: 10.1038/nature09534 (2010).
44 Iafrate, A. J. et al. Detection of large-scale variation in the human genome. Nature genetics 36, 949-951, doi:10.1038/ng1416 (2004).
45 Xu, H. et al. SgD-CNV, a database for common and rare copy number variants in three Asian populations. Human mutation 32, 1341-1349, doi:10.1002/humu.21601 (2011).

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Gut Enhancer Deletions Cause Severe Intractable Diarrhea

Distant-acting transcriptional enhancers are a predominant category of non-coding DNA in the human genome. However, the detection and functional interpretation of causative mutations affecting enhancers in human disorders remains challenging. Here are identified microdeletions of a non-coding sequence (intestine-critical region, ICR) on human chromosome 16p13.3 that cause inherited severe and intractable congenital diarrhea in affected infants. Transgenic mouse reporter assays show that the ICR is a transcriptional enhancer active in vivo during development of the gastrointestinal system. Targeted deletion of the ICR enhancer in mice cause symptoms recapitulating all major aspects of the human condition. Transcriptome analyses of human and mouse intestinal tissues reveal that the ICR deletion affects the expression of multiple genes, including strong down-regulation of gastrointestinal hormone peptides. Taken together, these results demonstrate that an enhancer deletion causes a severe congenital disorder and highlight the increasing potential for the discovery of disease-causing non-coding mutations as whole genome sequencing becomes routine in the clinic.

In this Example, it is demonstrated how the identification of non-coding deletions in a small number of patients is coupled to purpose-built mouse models which can be used to elucidate the regulatory basis of an inherited severe disease. It is also shown that mice carrying the non-coding deletion accurately recapitulate molecular and physiological phenotypes of the human disease condition, thus providing an animal model to explore the etiology of the human disorder.

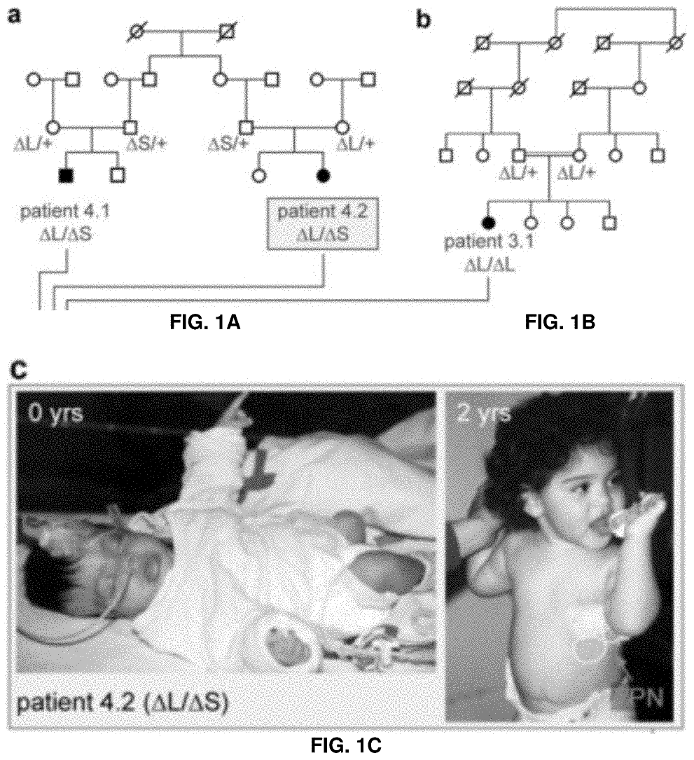

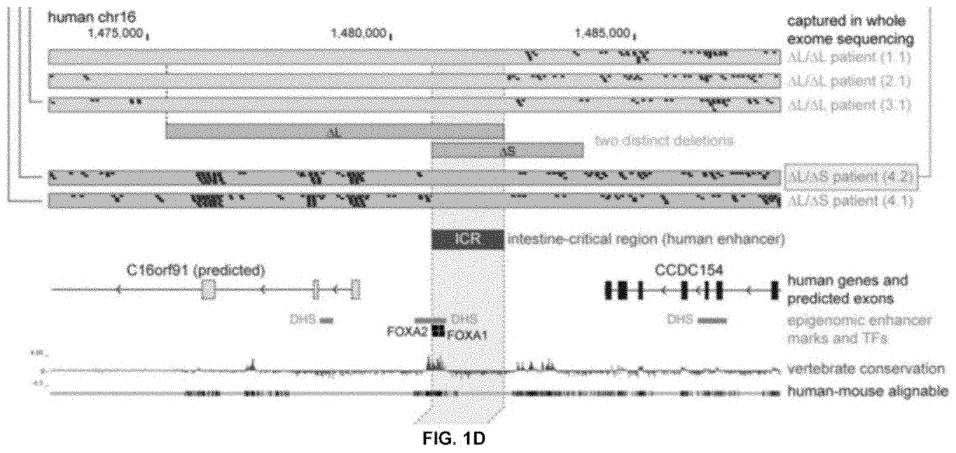

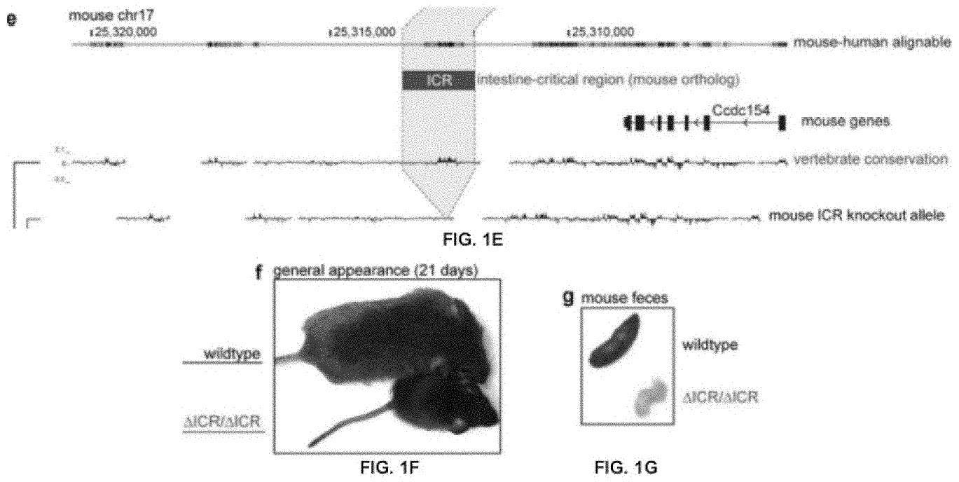

Congenital diarrhea disorders are a heterogeneous group of inherited diseases of the gastrointestinal tract starting within the first few weeks of life, often immediately after birth⁵⁻⁷. These disorders are often life-threatening, cannot be successfully treated, and affected individuals often depend on life-long parenteral nutrition (FIG. 1C) and in some cases small bowel transplantation⁸.

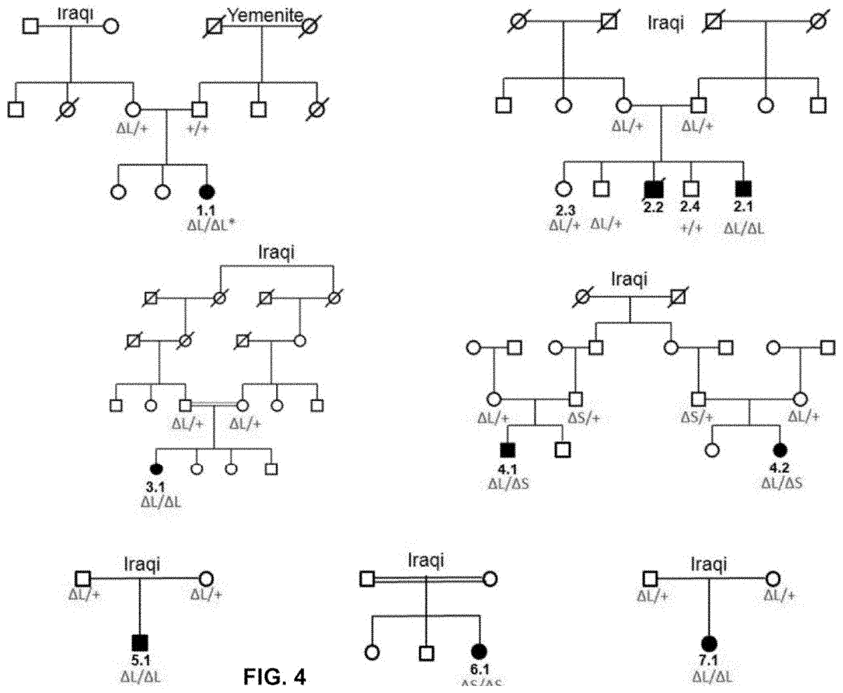

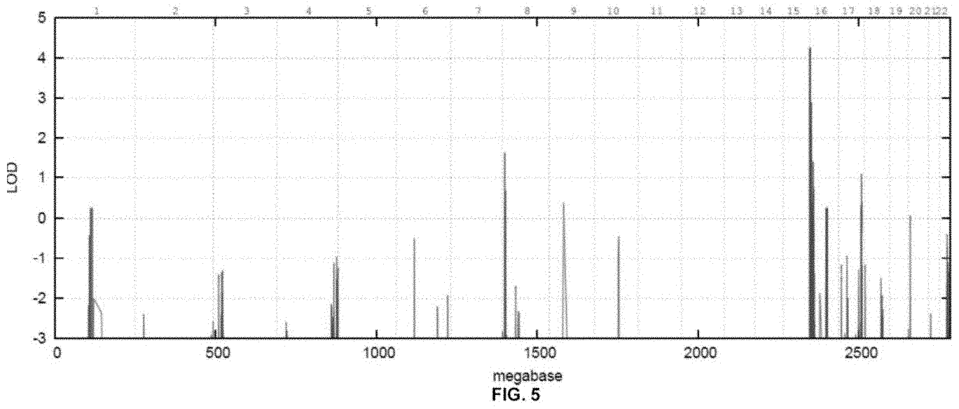

Eight patients from seven unrelated families of common ethnogeographic origin are studied with an autosomal recessive pattern of severe congenital malabsorptive diarrhea⁷ (FIGS. 1A and 1B; FIG. 4). While WES analysis reveal no rare exonic sequence variants with the appropriate patient segregation, whole genome linkage analysis and haplotype reconstruction detected a single significant telomeric linkage interval on chromosome 16 (LOD=4.26; FIG. 5).

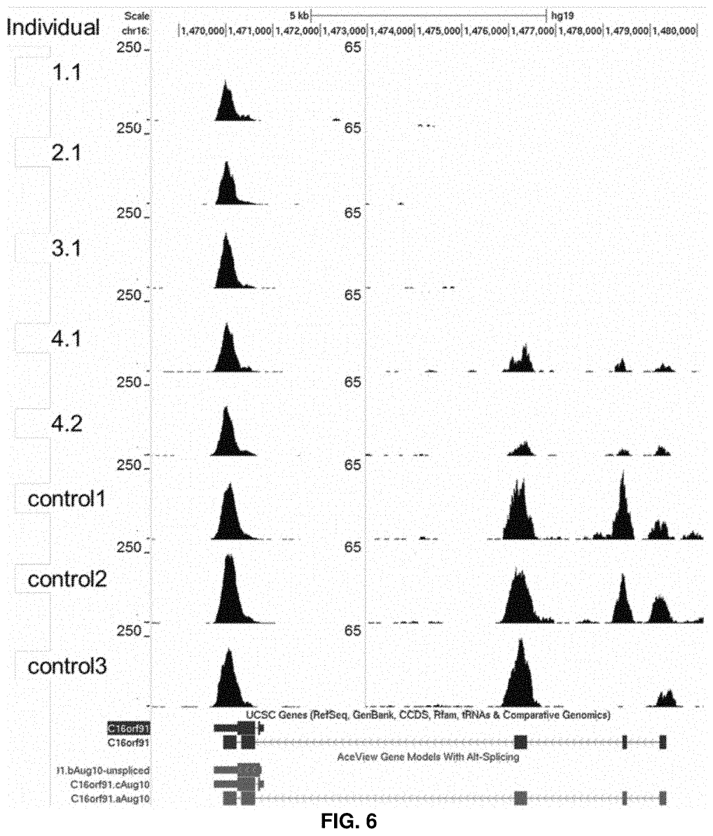

To identify possible structural genomic changes at this locus, all WES data sets, as well as WGS data from one of the patients are further examined. In WES data, an absence of coverage of three consecutive exons of a predicted transcript of C16ORF91 is observed in a subset of patients, suggesting the presence of a deletion (FIG. 1D, FIG. 6). Consistent with this observation, WGS data shows the deletion of a 7,013 bp segment, termed ΔL. PCR amplification and Sanger sequencing confirm the presence of a homozygous ΔL deletion in the patient examined by WGS, as well as most of the other patients examined (FIG. 4). No other structural changes or protein-coding mutations in the linkage interval are observed in WGS data from a ΔL/ΔL patient. Further scrutiny reveals that none of the three computationally predicted exons within the deleted interval are supported by quantitative RT-PCR (Methods), or by public transcription resources (UCSC genome browser, Illumina Body Map, ENCODE), providing a first line of evidence suggesting that a non-coding function may be affected by the deletion. Targeted PCR and sequencing of the locus show that two of the patients are compound heterozygous for ΔL along with a distinct allelic variant. This second variant, termed ΔS contains a 3,101 bp deletion that does not include any of the three hypothetical C16ORF91 exons but partially overlaps ΔL, defining a minimal sequence termed intestine-critical region (ICR) of 1,528 bp (FIG. 1D). All eight patients in this study show ΔS/ΔS, ΔS/ΔL or ΔL/ΔL genotypes, resulting in homozygous deletion of the ICR (FIG. 4). Neither of these deletions are found in several large control samples, including 200 ethnicity-matched controls and >3,000 WGS data sets from diverse sources. Taken together, these human genetic data strongly suggest that the ICR is non-coding and causes the congenital diarrhea phenotype.

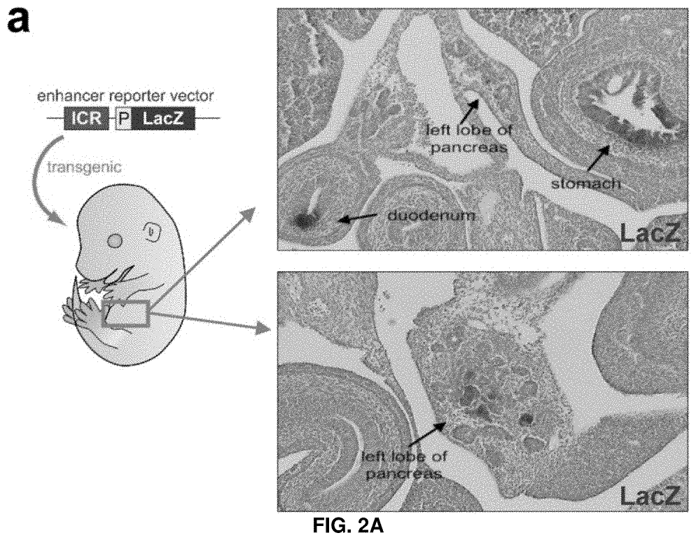

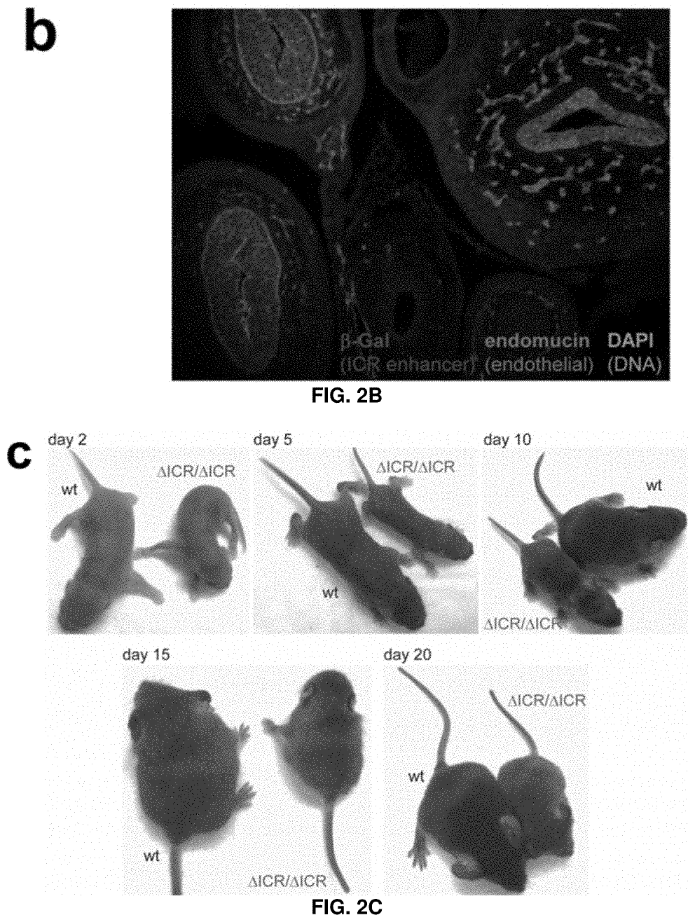

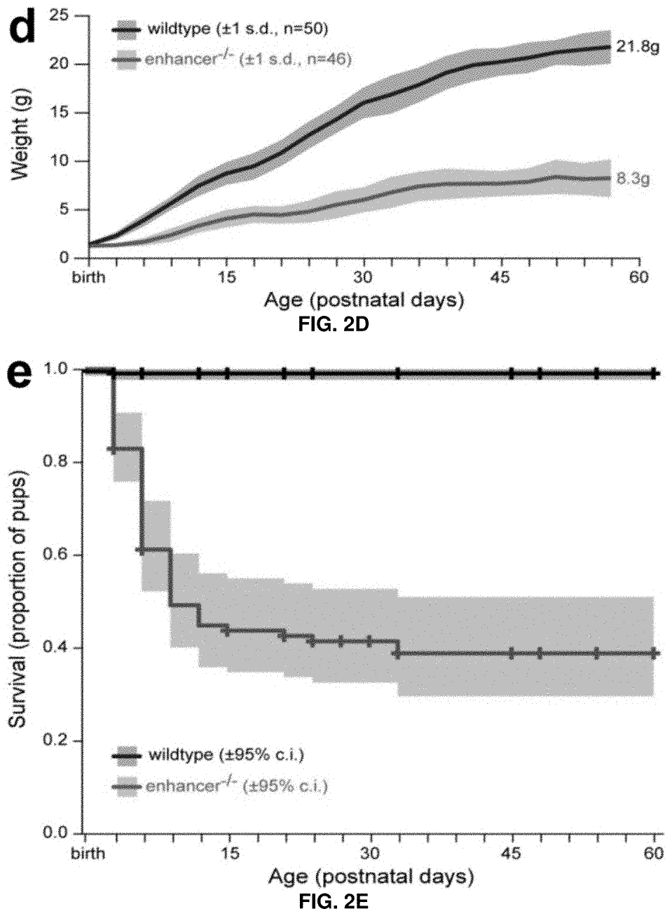

To explore possible non-coding functions of the ICR sequence, Encyclopedia of DNA Elements (ENCODE) data⁹ are examined. The interval contains a 400 bp region with high evolutionary conservation across vertebrates that shows CpG island and DNAse hypersensitivity signatures, and encompasses a cluster of multiple binding sites for transcription factors identified by ChIP-seq (FIG. 1D). The strongest ChIP-seq signal is observed for enhancer-interacting transcription factors FOXA1 and FOXA2^{10,11}, raising the possibility that the ICR is a distant-acting enhancer. To test this hypothesis, the enhancer activity of the minimal critical human interval is examined in a transgenic mouse enhancer assay¹². In transgenic embryos ranging from embryonic day (E) 11.5 to E14.5, robust and reproducible reporter activity is observed in the stomach, pancreas and duodenum (FIGS. 2A and 2B). All three of these organs contain many distinct enteroendocrine cell types that control gastrointestinal and metabolic function via hormone peptides¹³. These results support the notion that the ICR sequence deleted in congenital diarrhea patients contains an enhancer active in vivo in the developing digestive system, and may thus be directly linked to the disease etiology.

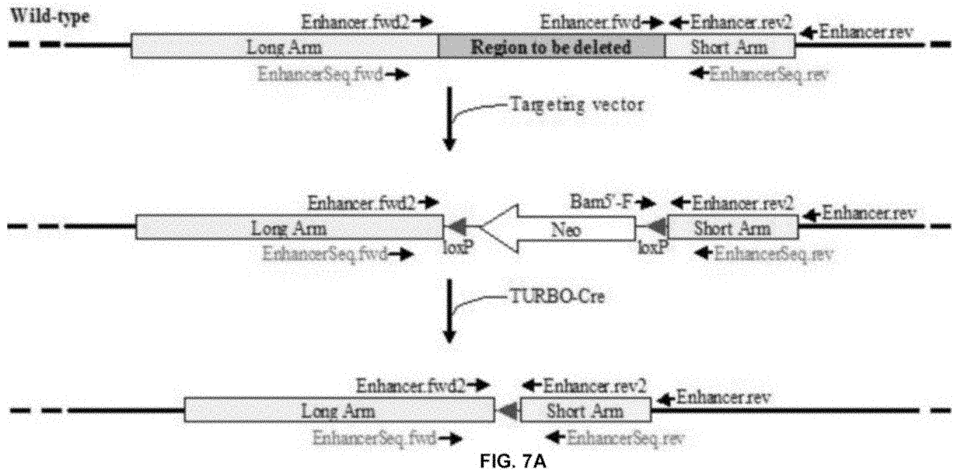

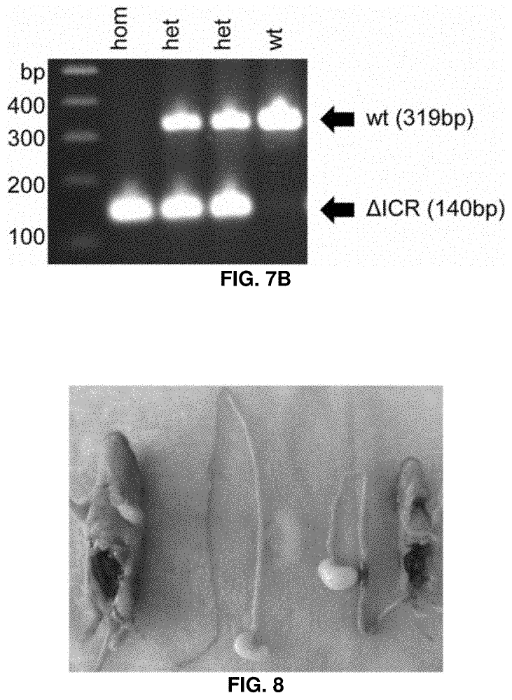

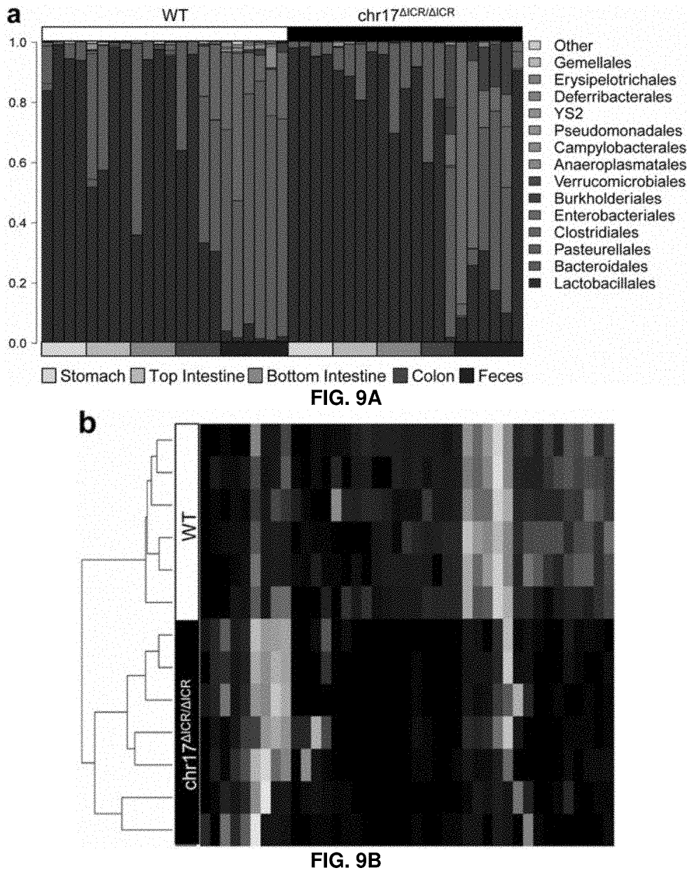

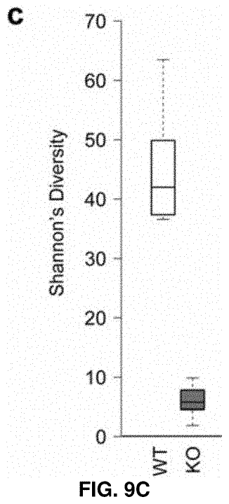

To examine if deletion of the minimal ICR sequence is sufficient to cause the in vivo phenotypes observed in human patients, a 1,512 bp mouse sequence orthologous to the human 1,528 bp ICR from the mouse genome is removed using homologous recombination in embryonic stem cells (FIG. 1E, FIGS. 7A and 7B). When heterozygous chr17^{+/ΔICR} mice are interbred, homozygous chr17^{ΔICR/ΔICR} offspring are born at the expected Mendelian frequency. At birth, the pups show no gross phenotypes and have normal suckling behavior. However, starting within the first few days of life, chr17^{ΔICR/ΔICR} mice display overall reduced size (FIG. 2C), low body weight (FIG. 2D) and substantially decreased survival (FIG. 2E). Only 40% of chr17^{ΔICR/ΔICR} mice survive to weaning at ˜20 days of age and by two months after birth, surviving chr17^{ΔICR/ΔICR} mice show a 60% reduction in weight compared to wild-type or heterozygous littermates. Examination of fecal pellets and internal organs reveal abnormal digestive tract function in chr17^{ΔICR/ΔICR} mice. The stomach content of chr17^{ΔICR/ΔICR} mice during the first weeks of life do not show gross deviations from wild-type controls in volume or appearance and consisted of normal amounts of milk. However, the intestinal content is abnormal, with pale undigested appearance, much softer consistency, and failure to form discrete fecal pellets (FIG. 1G; FIG. 8). Microscopic histological analysis of intestinal content and 16S rRNA-based sequence profiling of microbial communities in different intestinal compartments and feces identify substantial changes in the composition of the intestinal microbiome in chr17^{ΔICR/ΔICR} mice (FIG. 9A to 9C). These results indicate that deletion of the ICR enhancer in mice causes substantial disruption of intestinal function, consistent with the in vivo activity of the enhancer in the developing intestinal tract and recapitulating the congenital diarrhea phenotype observed in human patients carrying homozygous ICR deletions.

To explore the molecular basis of the phenotypes observed upon ICR deletion, possible changes in gene transcription in human and mouse digestive tract tissues are examined. Such changes may reflect dysregulation of direct target genes of the ICR enhancer, indirect downstream regulatory events, or the absence or general dysfunction of intestinal cell populations. RNA sequencing of duodenal and stomach biopsies obtained from a ΔL/ΔL patient are performed, as well as a non-diseased sibling. Among the genes showing the strongest down-regulation genome-wide in at least one of these tissues, eight encode gastrointestinal peptide hormones secreted by enteroendocrine cells¹⁴, and four have other relationships to gastrointestinal function (Table 1). Top 30 upregulated and downregulated genes, constructed with a threshold of X7 up or downregulation. These genes are selected by from a longer list in duodenal and stomach biopsies comparing affected to a sibling wild-type control. The fold changes are calculated as the expression ratio wild type/affected for down regulated genes and affected/wild type for up regulated genes.

Particularly pronounced changes are observed for five peptide hormones: gastric inhibitory polypeptide (GIP), motilin (MLN) and ghrelin (GHRL) in the duodenum and gastrin (GAST) and somatostatin (SST) in the stomach, all of which show >100-fold reduction in expression. In addition MBOAT4^{15,16}, a ghrelin-modifying enzyme, and ARX, a transcription factor controlling enteroendocrine celldevelopment¹⁷ and associated with syndromic congenital diarrhea^{18,19} show 20- to 30-fold down-regulation in the ΔL/ΔL small intestine. These results are consistent with abnormal development or function of enteroendocrine cells²⁰. Among the genes showing the largest increase in expression, eight are related to the gastrointestinal tract including gastrokines 1 and 2 (GKN1, GKN2), crucial for homeostasis of gastric epithelial cells and maintenance of gastric mucosa integrity²¹, pepsin precursor (PGA3) and motilin receptor (MLNR; Table 1). Quantitative RT-PCR of selected candidates including seven gastrointestinal peptide hormones and ARX confirmed their dysregulation in ΔL/ΔL samples. Consistent with these observations in human patients, RNA sequencing of a panel of mouse digestive tract biopsies taken at different stages of development show that nearly all of these genes are dysregulated in chr17^{ΔICR/ΔICR} mice. For the genes shown in Table 1, across all profiled mouse digestive tract tissues 121 of 191 valid comparisons show significant changes in expression (p<0.05), the vast majority of which (105 of 121; 87%) is in the same direction as in human biopsies. Together, these results are consistent with major disruptions of normal intestinal physiology in chr17^{ΔICR/ΔICR} humans and mice and highlight the close resemblance between the human disease condition and the mouse knockout model.

TABLE 1
Significant expression changes in human and mouse intestinal tissue. A selection of down- and up- regulated
genes associated with gastrointestinal tract function are provided. Fold changes are calculated as the
expression raytio of non-affected human or wild-type mice over homozygous ΔICR/ΔICR patients or
mouse littermates. n.e., not expressed. n/a, not applicable. Fold-change and p- value for the mouse
tissue with quantitatively strongest genotype-dependent requlation in same direction as human tissue
shown. p-values are Bonferroni-corrected for multiple hypothesis testing across 16 mouse tissues.

| | | Fold Changes | | | | |
|---|---|---|---|---|---|---|
| | | human small | human | | | |
| Gene | Description | intestine | stomach | mouse | P | mouse tissue |

Down-Regulated in human patients/chr17^{Δ}^{^{V}}^{/Δ}^{^{V}} mice

| | | | | | | |
|---|---|---|---|---|---|---|
| SST | somatostatin | 10 | 683 | 36 | <0.01 | colon/rectum (P1) |
| GIP | gastric inhibitory peptide | 277 | n.e. | 768 | <0.001 | intestine (P5) |
| MLN | motilin | 206 | n.e. | — | — | (no mouse ortholog) |
| GHRL | ghrelin/obestatin prepropeptide | 125 | 5.2 | 896 | <0.001 | stomach (P10, bottom) |
| CEL | carboxyl ester lipase | 1.1 | 135 | 144 | <0.001 | intestine (P1, top) |
| ARX | aristaless related homeobox | 30 | 6 | 23 | <0.05 | stomach (P10, bottom) |
| PYY | peptide YY | 25 | n.e. | 223 | <0.001 | rectum (P5) |
| MBOAT4 | ghrelin O-acyltransferase | 22 | 1.4 | 9.4 | <0.01 | stomach (P20, bottom) |
| NTS | neurotensin | (0.62) | 15 | 674 | <0.001 | intestine (P1, bottom) |
| GAST | gastrin | 11 | 123 | 52 | <0.001 | stomach (P5) |
| CCK | cholecystokinin | 8.2 | 6.7 | 109 | <0.001 | intestine (P5, top) |
| SLC26A7 | solute carrier family 26, member 7 | 7.4 | 2.9 | 6.2 | (>0.05) | stomach (P1) |

Up-Regulated in human patients/chr17^{Δ}^{^{V}}^{/Δ}^{^{V}} mice

| | | | | | | |
|---|---|---|---|---|---|---|
| GKN1 | gastrokine 1 | 256 | n.e. | 25 | <0.001 | colon (P5) |
| PGA3 | pepsinogen A3 | 113 | 6.96 | — | — | (no mouse ortholog) |
| GKN2 | gastrokine 2 | 60 | (0.81) | 22 | <0.001 | colon (P5) |
| DUOX2 | dual oxidase 2 | 51 | (0.34) | 19 | <0.001 | intestine (P1, top) |
| RBP2 | retinol binding protein 2 | (0.89) | 20 | 8 | <0.001 | colon (P5) |
| REG1B | regenerating islet-derived 1 beta | 14 | n.e. | 1946 | <0.001 | stomach (P10, bottom) |
| MLNR | motilin receptor | 1.0 | 12 | — | — | (no mouse ortholog) |
| ATP4B | ATPase, H+/K+ exchanging, beta | 7.6 | 4.5 | 345 | <0.001 | intestine (P1, top) |

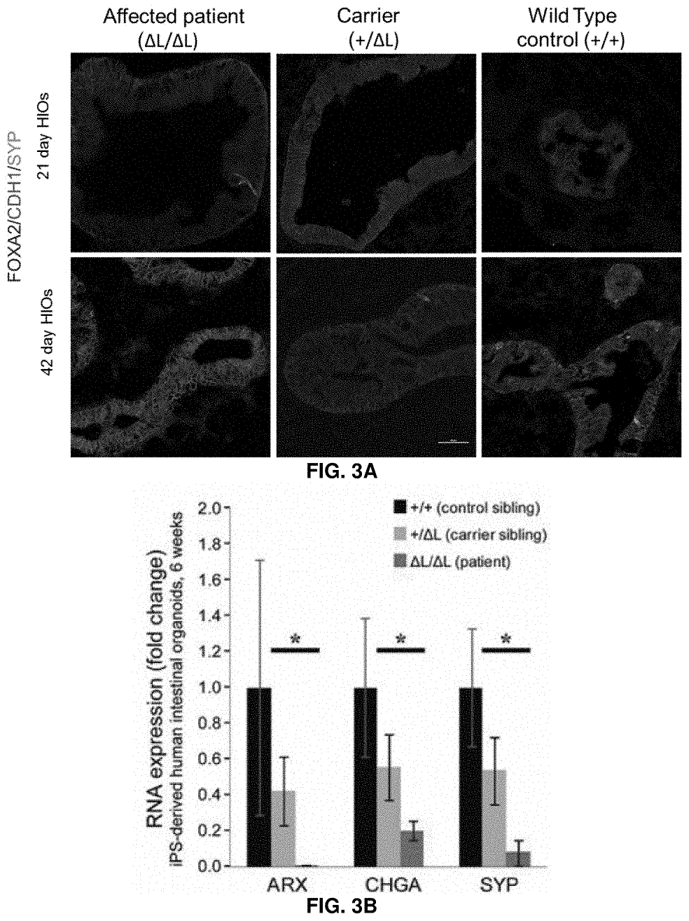

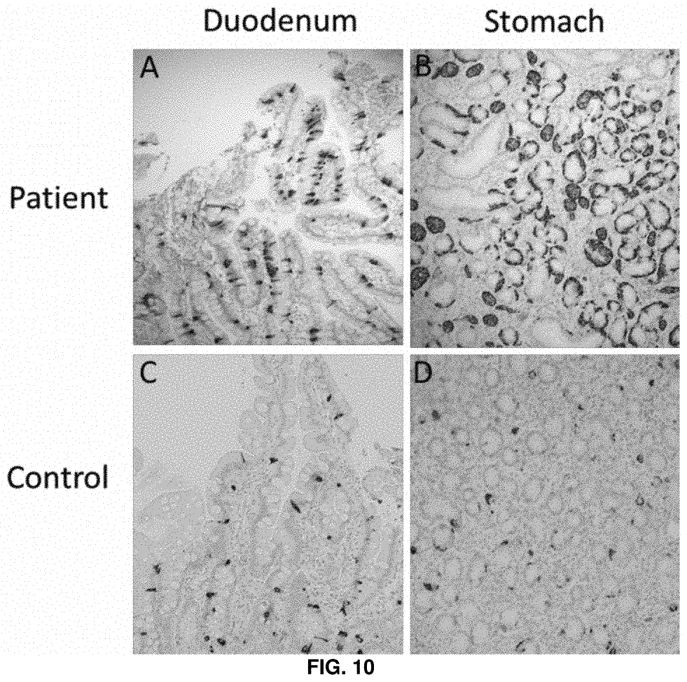

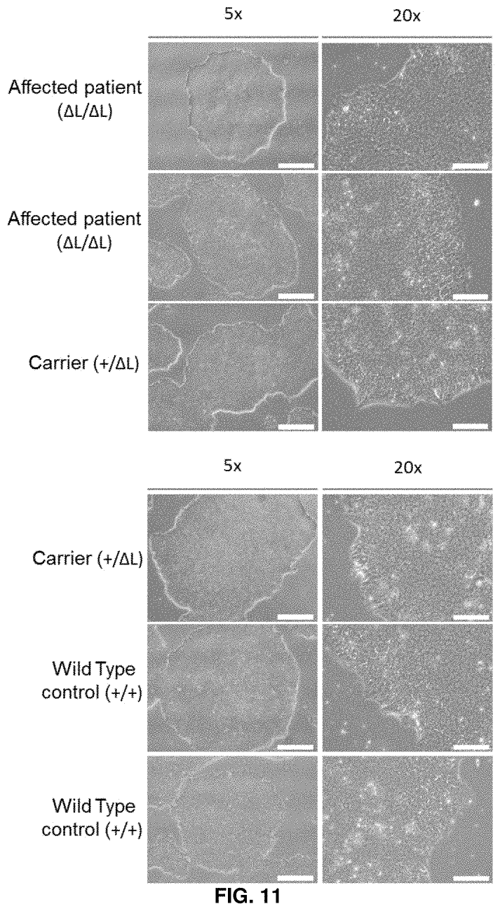

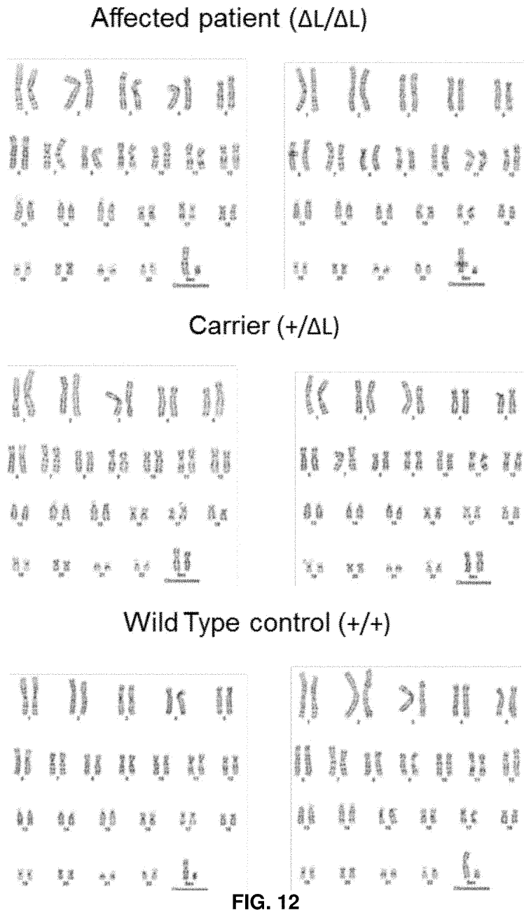

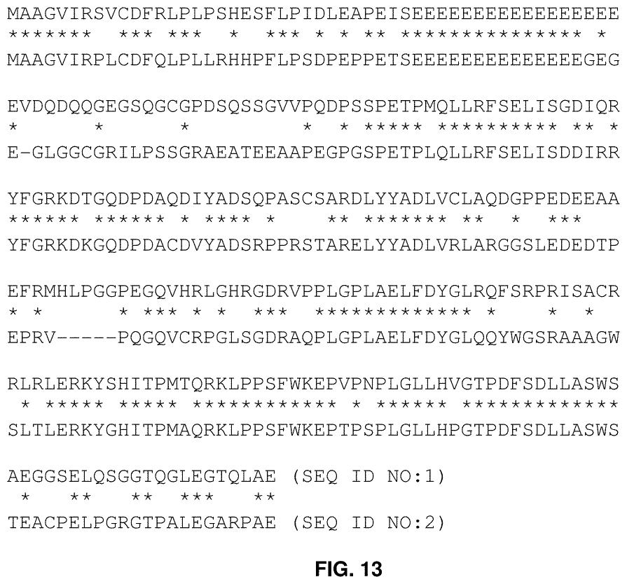

To further explore the pathophysiology associated with ICR deletions, biopsies obtained from two ΔL/ΔL homozygous patients are subjected to immunohistochemical staining with chromogranin A (CHGA), an early marker of enteroendocrine cell development. Increased immunoreactivity, as compared to healthy controls, is seen in the duodenal villi and stomach pyloric mucosae, a hyperplastic change that further supports that ICR deletions cause abnormal development of enteroendocrine cells (FIG. 10). To investigate whether ICR deletions cause abnormalities in the development of human enteroendocrine cells, induced pluripotent stem cell (iPSC) lines are generated from a ΔL/ΔL patient, a heterozygous +/ΔL sibling, and an unaffected +/+ sibling and differentiated them into human intestinal organoids (HIOs) (FIGS. 11 and 12). Differentiation of iPSCs into intestinal tissues in vitro is highly similar to development of the embryonic intestine, and after 21 and 42 days in culture, HIOs from all three genotypes formed an intestinal epithelium that expressed CDH1, FOXA2 (FIG. 3A) and CDX2 (data not shown). Analysis of enteroendocrine cells with the markers Synaptophysin (SYP, FIG. 3A) and Chromogranin A (CHGA, not shown) indicate that these cells are more readily detected in the ΔL/ΔL iPSC HIOs than in the HIOs generated from carrier or control iPSC lines after 21 days in culture, similar to biopsy specimens. In contrast, the number of enteroendocrine cells at the later (42 day) time point is severely reduced in ΔL/ΔL HIOs. These results are confirmed by quantitative RT-PCR where ΔL/ΔL HIOs show a substantial decrease in the expression of enteroendocrine markers CHGA, SYP, as well as ARX (FIG. 3B). These results suggest that specification of enteroendocrine cells during development and in adults is normal or even precocious in ΔL/ΔL patients, but that later stages of development and differentiation are impaired. It is noted that patient biopsies show increased immunoreactivity of CHGA (FIG. 11), which may indicate that in vivo these tissues acquire a steady state, whereas the in vitro HIO model recapitulates the initial emergence of enteroendocrine cells during embryonic development²⁰.

The involvement of distant-acting regulatory regions in human diseases remains poorly understood and few cases of disease-causing variations that affect transcriptional enhancers have been documented²²⁻²⁶. Only one of these examples constitutes a complete deletion of an enhancer²⁴ and it remains unclear if deletion of the homologous sequence in mice produces a phenotype mimicking the human condition. It is shown that a deletion of a developmental enhancer sequence is the cause of a severe, recessively inherited gastrointestinal disease. Enhancer activity is highly tissue-specific, and the tissues with enhancer activity in vivo are consistent with the gastrointestinal disease etiology. The observed molecular and physiological phenotypes suggest that the enhancer deletion affects normal development of enteroendocrine cells and thereby normal enteroendocrine hormone secretion. This is supported by the striking phenotypic similarity between chr17^{ΔICR/ΔICR} mice and mice with an intestinal-specific deletion of Neurog3, a proendocrine transcription factor required for development of enteroendocrine cells²⁷. Since chr17^{ΔICR/ΔICR} mice resemble human patients homozygous for ICR deletions in all disease aspects examined in this study, these mice are likely to provide an accurate model for studying the human condition and exploring therapeutic interventions. Beyond congenital diarrhea, the results highlight the potential role that distant-acting regulatory elements may play in the pathology of other Mendelian diseases. While WGS approaches identify increasing numbers of disease-associated non-coding variants, their functional interpretation remains challenging. This example demonstrates the importance of detailed experimental follow-up of such findings through in vivo models, an approach that will benefit from the emerging suite of highly efficient genome editing tools²⁸.

### Methods

Subjects:

IDIS patients are recruited at Schneider and Sheba medical centers in Israel. The study is conducted in accordance with the Declaration of Helsinki, and all subjects and their family members had given informed consent for genetic testing and reproduction of patient photos.

Exome Sequencing and Variants Identification:

Exome sequencing is performed using Agilent SureSelect Human All Exon technology (Agilent Technologies, Santa Clara, Calif.). The captured regions are sequenced using Genome Analyzer IIx (Illumina, Inc. San Diego, Calif.). The resulting reads are aligned to the reference genome (build 37) using the Burrows-Wheeler Alignment (BWA) tool²⁹. 70× coverage, where a base is considered covered if ≥5 reads spanned the nucleotide is obtained. Genetic differences relative to the reference genome are identified by the SAMtools variant calling program³⁰, which identifies both single nucleotide variants and small insertion-deletions (indels). Finally, the Sequence Variant Analyzer software (SVA)³¹ is used to annotate all identified variants. For comparison to controls 1000 samples are subjected to exome or whole genome sequencing at the Center for Human Genome Variation (CHGV, Duke University, NC, USA), dbSNP, 1000 genomes, and NHLBI GO Exome-sequencing Project.

Whole Genome Sequencing:

WGS of individual 2.1 is performed at CHGV, using the Illumina HiSeq platform (Illumina, Inc. San Diego, Calif.) and analyzed as described for exome data. 275 CHGV whole-genome sequenced, unrelated samples are used as controls. To detect copy number variants from WGS the Estimation is used by read depth with single-nucleotide variants (ERDS) tool³².

Biopsy Collection:

Subjects underwent gastro-duodenoscopy following Institutional Review Board (IRB) approval (No. 9881-12-SMC) at Sheba Medical Center, and written informed consent of the patients and family members.

RNA Extraction from Biopsies:

RNA isolation from frozen biopsies is performed using TRI Reagent® method (Sigma-Aldrich Inc.) according to the manufacturer's instructions or by Qiagen RNeasy Mini Kit (Qiagen, Valencia, Calif., USA). Integrity of the samples is measured for concentration and purity using NanoDrop® Spectrophotometer (Nanodrop Technologies, Wilmington, Del., USA).

RNA Sequencing of Human Samples:

Total RNA is prepared according to the Illumina RNA-seq protocol: briefly, globin reduction, polyA enrichment, chemical fragmentation of the polyA RNA, cDNA synthesis, and size selection of 200 bp cDNA fragments are performed. Next, the size-selected libraries are used for cluster generation on the flow cell and prepared flow cells are run on the Illumina HiSeq2000 (Illumina, Inc. San Diego, Calif.). A total of 74.18 million paired-end reads of a 100 bp are obtained for the affected sample and 72.53 million reads to the healthy sample. Reads are aligned to the human genome (NCBI37/hg19) using Tophat v2.0.4³² with the default parameters. Gene expression quantification is performed with cuffdiff³³ using the Illumina iGenome project UCSC annotation file as a reference.

Quantitative Real-Time Reverse Transcriptase Polymerase Chain Reaction (qPCR):

RNA extracted from the biopsies is used for qPCR expression analyses. qPCR is performed using TaqMan® Gene Expression Assays (Applied Biosystems, Foster City, Calif., USA) using the Applied Biosystems StepOnePlus (Applied Biosystems). From 1 μg of biopsy RNA, cDNA is synthesized using the SuperScript® First-strand Synthesis System for RT-PCR (Invitrogen, Carlsbad, Calif., USA) according to the manufacturer's instructions. A total of 20 μl of cDNA is added with 30 μl of water to 50 μp of TaqMan® universal PCR Master Mix (Applied Biosystems) and the resulting 100 μl reaction mixtures are loaded onto a 96-well PCR plate. 14 different TaqMan® Gene Expression Assay are used including three housekeeping genes with the following assays IDs: Hs00757713_m1 (MLN), Hs01074053_m1 (GHRL), Hs00175048_m1 (NTS), Hs00356144_m1 (SST), Hs00174945_m1 (PYY), Hs01062283_m1 (GAST), Hs00292465_m1 (ARX), Hs00174937_m1 (CCK), Hs00175030_m1 (GIP), Hs00219734_m1 (GKN1), Hs00699389_m1 (GKN2).

The housekeeping genes are HMBS (Hs00609297_m1), ACTB (Hs99999903_m1) and GAPDH (Hs99999905_m1). Reference cDNA samples are synthesized using 200 ng of RNA from RNA extracted from stomach and duodenum tissues of two healthy controls (BioCat GmbH, Heidelberg, Germany) for use in the normalization calculations. Quantitative RT-PCR for expression analysis on the missing exons in C16ORF91 is done using cDNA extracted from the Human Digestive System MTC™ Panel (Clontech Laboratories, Inc. Mountain View, Calif.).

Serum Collection:

Whole blood is withdrawn into a Vacutainer serum tube without anti-coagulant. The blood is immediately treated with 1 μM AEBSF (protease inhibitor) and remains at room temperature for 30 min to clot before centrifugation (15 min at 2500 rpm at 4° C.).

ELISA:

Serum hormone levels are determined using sandwich ELISA technique performed by the following commercial kits according to the manufacturer's instructions. Human Ghrelin (Total) ELISA COLD PACKS (Millipore, USA), Human PYY (Total) ELISA Kit (Millipore), and Human gastric inhibitory polypeptide (GIP) ELISA Kit (ENCO).

Linkage Analysis and Homozygosity Mapping:

Genome-wide SNP genotyping from DNA of 6 affected children and 22 relatives from families 1-5 is performed using the Illumina HumanCytoSNP-12v2-1_H, according to the manufacturer's recommendations (Illumina, Inc. San Diego, Calif.) in conjunction with SNP genotypes retrieved from whole exome data. For linkage studies 35,845 informative equally spaced SNP markers are chosen after filtering for Mendelian errors and unlikely genotypes. Genotypes are examined with the use of a multipoint parametric linkage analysis and haplotype reconstruction for an autosomal recessive model with complete penetrance and a disease allele frequency of 0.001 as previously described³⁴. Homozygosity mapping is performed using PLINK³⁵ with the default parameters (length 1000 kb, SNP(N) 100, SNP density 50 kb/SNP, largest gap 1000 kb).

Deletion Analysis:

Boundaries for the two deletion alleles are determined by PCR using amplified DNA and Sanger sequencing. The specific primers are used amplifying across both deletions and inside the overlap region for the two deletions are reported in Table 2. In parallel, polymorphic markers are used that are identified by electronically screening genomic clones located on Chr16 0.86-2.8 Mb. Primers are designed with the Primer3 software (website for: frodo.wi.mit.edu/cgi-bin/primer3/primer3_www.cgi/ from the Whitehead Institute, Massachusetts Institute of Technology, and Cambridge, Mass.). The specific primers used are reported in Table 3. Amplification of the polymorphic markers is performed in a 25-μl reaction containing 50 ng of DNA, 13.4 ng of each primer, and 1.5 mM dNTPs in 1.5 mM MgCl₂ PCR buffer with 1.2 U Taq polymerase (Bio-Line, London, UK). After an initial denaturation of 5 minutes at 95° C., 30 cycles are performed (94° C. for 2 minutes, 56° C. for 3 minutes, and 72° C. for 1 minute), followed by a final step of 7 minutes at 72° C. PCR products are electrophoresed on an automated genetic analyzer (Prism 3100; Applied Biosystems, Inc. [ABI], Foster City, Calif.). The breakpoints coordinates are: ΔL—chr16: 1475365-1482378, ΔS—chr16:1480850-1483951, with an overlapping region at chr16: 1480850-1482378 (ICR).

TABLE 2
Primers for determining deletion boundaries by PCR. The
primers Del S F, IN DEL S F, Del S R, Del L F, HL-FN, and
Del L R are SEQ ID NOs: 17-22, respectively.

| Primer name. | Forward/Reverse primer (5′→3′) | TM° C. |
|---|---|---|
| Del S F (5′→3′) | CAT GTG CCG CAT CTC TGG AC | 59 |
| IN DEL S F (5′→3′) | GGA CCG TGG AGT GTT TGT GC | 59 |
| Del S R (5′→3′) | CAG TGG AGA TGG TCA TGG CTG T | 59 |
| Del L F (5′→3′) | TCT TCC TCC TCC GAA GTC TCT | 59 |
| HL-Fn (5′→3′) | AAA CAG GTG CCT CTG TTG ACA C | 59 |
| Del L R (5′→3′) | CAA TCT CAA CTC ACT GCA ACC TCT | 59 |

TABLE 3
Primers for polymorphic markers. The primers AC098805 fwd and rev, are
SEQ ID Nos: 23 and 24, respectively. The primers AL023882 fwd and rev,
are SEQ ID Nos: 25 and 26, respectively. The primers AC009041 fwd and rev,
are SEQ ID Nos: 27 and 28, respectively. The primers AC120498 fwd and rev,
are SEQ ID Nos: 29 and 30, respectively. The primers AC012180 fwd and rev,
are SEQ ID Nos: 31 and 32, respectively. The primers AC005363 fwd and rev,
are SEQ ID Nos: 33 and 34, respectively. The primers AL032819 fwd and rev,
are SEQ ID Nos: 35 and 36, respectively.

| | Location | | |
|---|---|---|---|
| Marker | Mbp | Forward primer (5′→3′) | Reverse primer (5′→3′) |
| AC098805 | Ch16-2.3 | GCCCGGTCATAAATTGTTGTAT | TCTGCCAAAAGTCTAGGTGTG |
| AC023882 | Ch16-0.87 | GCCTGTGGATGGTGAATTTT | ACTACAGGTGCCACCACCAC |
| AC009041 | Ch16-1.1 | CACGCTCGCACTCGTATG | CCTGACGCTCAGCTAGGAAG |
| AC120498 | Ch16-1.25 | ATGGCCCCTGTATGTCTTTTC | AAACAACAGCTGGGCATGGT |
| AC012180 | Ch16-1.81 | ATCCTCGTGCTATGAACAGACA | GAGCACTATTCTGCCTCCCATA |
| AC005363 | Ch16-1.98 | CCATAGTTTCTAACCCTCAGCA | ATGGAATGTTAGCATTGGCTCT |
| AL032819 | Ch16-1.45 | TGA TGA GCT CTG AAA AGC G | GAA CCT GCC CCT CTG TCT C |

Mouse Transgenic Assays:

The candidate sequence containing the expected enhancer (chr 16: 1479875-1480992) is PCR amplified from human genomic DNA and, using Gateway (Invitrogen) cloning, is cloned into an Hsp68-lacZ vector containing a minimal Hsp68 promoter coupled to a lacZ reporter gene. The construct is microinjected into fertilized FVB/N mouse oocytes, which are implanted into pseudopregnant foster females and embryos are collected at E11.5 through E14.5. Enhancer reporter activity is determined by X-gal staining to detect 3-galactosidase activity. Only patterns observed in at least three different embryos resulting from independent transgenic events are considered reproducible positive enhancers.

Generation of Enhancer Null Mice:

Homologous arms are generated by PCR (see Suppl. Table S5 for primers) and cloned into ploxPN2T vector, which contains neomycin resistant cassette flanked by loxP for positive selection, and an HSV-tk cassette for negative selection. Constructs are linearized and electroporated (20 μg) into W4/129S6 mouse embryonic stem cells (Taconic). The electroporated cells are selected under G418 (150 μg/ml) and 0.2 μM FIAU for a week. Surviving colonies are picked and expanded on 96-well plates, screened both by PCR and sequencing with primers outside but flanking the homologous arm. Clones that are correctly targeted are electroporated with 20 μg of the Cre recombinase-expressing plasmid TURBO-Cre. TURBO-Cre is provided by Dr. Timothy Ley of the Embryonic Stem Cell Core of the Siteman Cancer Center, Washington University Medical School.

Clones positive for Neo removal are screened by PCR and checked for G418 sensitivity. PCR products covering the deleted region and part of homologous arms are gel purified and sequenced to confirm the deletion of the ICR enhancer.

Correctly targeted clones are subsequently injected into C57BL/6J blastocyst stage embryos. Chimeric mice are then crossed to C57BL/6J mice (Charles River) as well as 129S6/SvEvTac (Taconic) to generate heterozygous enhancer null mice, followed by breeding of heterozygous littermates to generate homozygous enhancer null mice.

Genotyping of Enhancer Null Mice:

Genomic DNA is extracted from a 0.2 to 0.3-cm section of tail that is incubated overnight in lysis buffer (containing 100 mM Tris-HCl pH 8.5, 5 mM EDTA, 0.2% SDS, 200 mM NaCl and 50 μg Proteinase K) at 55° C. Genotyping is carried out using standard PCR techniques (see Table 4 for primers). One to two microliters of 50- to 100-fold diluted tail lysate is used in a 20 μl PCR containing 200 μM dNTP, 1.5 mM MgCl₂, 5 pmole of each forward and reverse primer and 0.5 U of Taq polymerase.

TABLE 4
Primers for generating and assessing ICR deletion in mouse embryonic stem
cells. The primers hs2295SA fwd and rev, are SEQ ID Nos: 37 and 38, respectively.
The primers hs2295LA fwd2 and rev, are SEQ ID Nos: 39 and 40, respectively. The
primers Bam5′-F and hs2295 rev, are SEQ ID Nos: 41 and 42, respectively. The primers
hs2295seq fwd and rev, are SEQ ID Nos: 43 and 44, respectively. The primers hs2295
fwd, fwd2 and rev2, are SEQ ID Nos: 45-47, respectively.

| Primer Name | Sequence | Product Size (bp) | Note |
|---|---|---|---|
| hs2295SA.fwd | ATCCAGCACACCCTCAGCTTTAACTAGTC | 1.738 | Short arm |
| hs2295SA.rev | CATTCTTTGGTCACATACAGGTGGGACCTT | | |
| hs2295LA.fwd2 | AGGTATGGTGGGAGATGGGGTAGTCA | 7.199 | Long arm |
| hs2295LA.rev | AGCCATGTCTAGGCTCCAAAGTGAGAAC | | |
| Bam5′-F | TTGGCTGGACGTAAACTCCTCTTCAG | 1.477 | PCR |
| hs2295.rev | CTAGTCCTCACACCCAGCTCTTTCAA | | screening |
| | | | targeting |
| | | | event |
| hs2295seq.fwd | CCTAGAACTTGCTATATAAACTGGACAAGC | Wt-2.456: | Sequencing |
| hs2295seq.rev | GTGAAGCGCTGGACGGAGAGATAATCAGTA | KO (+Neo)-2.987: | verfication |
| | | KO (-Neo)-1.027. | of knock- |
| | | | out clones |
| hs2295.fwd | GTGTCTTCTCTGTCCTCCTGGAGTCA | Wt-hs2295.fwd/ | Primers for |
| hs2295.fwd2 | GTTCTCACTTTGGAGCCTAGACATGGCT | hs2295.rev2. 319 | genotyping |
| hs2295.rev2 | GACTAGTTAAAGCTGAGGGTGTGCTGGAT | bp: | |
| | | Del-hs2295.fwd2/ | |
| | | hs2295.rev2. 140 | |
| | | bp. | |

RNA Sequencing of Mouse Tissues:

Total RNA is extracted from different intestinal regions and stomach of mice at E11.5, P1, P5, P10, P15 and P20 using TRIzol® Reagent (Invitrogen). RNAseq libraries are then constructed using Illumina TruSeq Stranded Total RNA Sample Preparation Kit following manufacture's recommendation. The libraries are sequenced using a 50 bp single end strategy with four samples per lane on an Illumina HiSeq instrument and data is analyzed using the same protocols as described for human, though with the mm9 mouse reference and Illumina iGenome project mouse genome annotation data.

16S Amplicon Analysis (iTags) of Microbial Community Diversity:

Feces and gut content samples are collected from chr17^{ΔICR/ΔICR} mice and wt littermates. DNA is extracted from these samples using PowerFecal® DNA Isolation Kit (MO Bio Laboratories). V4 16S regions are amplified from the DNA samples using barcoded primers and 5 PRIME™ HotMasterMix™ (Fisher Scientific) as previously described³⁶. Amplicons are pooled in equal amount, purified with AMPureXP® magnetic beads (Beckman Coulter), and sequenced.

Histological Analysis of Human Biopsies:

FFPE blocks are sectioned at a thickness of 4 μm and a positive control is added on the right side of the slides. All immunostainings are fully calibrated on a Benchmark XT staining module (Ventana Medical Systems Inc., USA). Briefly, after sections are dewaxed and rehydrated, a CC 1 Standard Benchmark XT pretreatment for antigen retrieval (Ventana Medical Systems) is selected for all immunostainings: Chromogranin A (1:500, Dako, Denmark), and Synaptophysin, (1:200, Life Technologies, Invitrogen, USA). Detection is performed with iView DAB Detection Kit (Ventana Medical Systems Inc., USA) and counterstained with hematoxylin (Ventana Medical Systems Inc., USA). After the run on the automated stainer is completed, slides are dehydrated in ethanol solutions (70%, 96%, and 100%) for one minute each. Sections are then cleared in xylene for 2 minutes, mounted with Entellan and cover slips are added. Chromogranin A and Synaptophysin show cytoplasmic staining.

Generation of Induced Pluripotent Stem Cells (iPSCs) from Patient Lymphocytes:

Whole blood is isolated by routine venipuncture from patient 2.1 and two healthy siblings (2.3-heterozygous carrier, 2.4-unaffected WT) at Sheba Medical Center in Israel, in preservative-free 0.9% sodium chloride containing 100 U/mL heparin. Blood is then shipped overnight to Cincinnati Children's Hospital Medical Center for iPS cell generation. Peripheral blood mononuclear cells (PBMCs) are isolated from whole blood by Ficoll centrifugation as previously described³⁷ and are used to derive iPSCs. Briefly, PBMCs are cultured for 4 days in DMEM containing 10% FCS, 100 ng/ml SCF, 100 ng/ml TPO, 100 ng/ml IL3, 20 ng/ml IL6, 100 ng/ml Flt3L, 100 ng/ml GM-CSF, and 50 ng/ml M-CSF (Peprotech). Transduction using a polycistronic lentivirus expressing Oct4, Sox2, Klf4, cMyc and dTomato is performed³⁸ following the second day of culture in this media. Transduced cells are then cultured for an additional 4 days in DMEM containing 10% FCS, 100 ng/ml SCF, 100 ng/ml TPO, 100 ng/ml IL3, 20 ng/ml IL6, and 100 ng/ml Flt3L. Media is changed every other day. PBMCs are then plated on 0.1% gelatin-coated dishes containing 2×10⁴ irradiated MEFs/cm² (GlobalStem, Rockville, Md.), and is cultured in hESC media containing 20% knockout serum replacement, 1 mM L-glutamine, 0.1 mM β-mercaptoethanol, 1× non-essential amino acids, and 4 ng/ml bFGF until iPSC colony formation. Putative iPSC colonies are then manually excised and re-plated in feeder free culture conditions consisting of matrigel (BD BioSciences, San Jose, Calif.) and mTeSR1 (STEMCELL Technologies, Vancouver, BC). Lines exhibiting robust proliferation and maintenance of stereotypical human pluripotent stem cell morphology are then expanded and cryopreserved before use in experiments. Standard metaphase spreads and G-banded karyotypes are determined by the CCHMC Cytogenetics Laboratory.

Differentiation of iPSCs into Intestinal Organoids:

The differentiation of induced human pluripotent stem cells is performed as previously described³⁹⁻⁴¹ with minor modifications. Briefly, two clonal iPSC lines from each donor are dispase passaged into a matrigel coated 24 well tissue culture plate and cultured for 3 days in mTeSR1. Following definitive endoderm differentiation, the monolayers are treated for 4 days with RPMI medium 1640 (Gibco) containing 2% defined fetal calf serum, 1× non-essential amino acids, 3 μM CHIR99021 (Stemgent) and 500 ng/mL rhFGF4 (R&D Systems) to induce hindgut spheroid morphogenesis. After the 4^{th} day, “day 0” HIOs are collected and embedded in matrigel matrix and cultured in Advanced DMEM/F12 (Gibco) containing 100 U/mL penicillin/streptomycin (Gibco), 2 mM L-Glutamine (Gibco), 15 mM HEPES (Gibco), N2 Supplement (Gibco), B27 Supplement (Gibco), and 100 ng/mL rhEGF (R&D Systems) for up to 42 days, splitting, passaging, and changing the media periodically.

HIOs collected for immunofluorescence analysis are fixed in 4% paraformaldehyde for 1-2 h at room temperature, washed overnight at 4° C. in PBS, and embedded in O.C.T. Compound (Sakura). Sections 8-10p thick are incubated with primary antibodies overnight at 4° C. in 10% normal donkey serum/0.05% Triton X-100-PBS solution and subsequently incubated with secondary antibodies for 1 h at room temperature. The primary antibodies used are: FoxA2 (1:500; Novus), E-Cadherin (1:500; R&D Systems), Synaptophysin (1:1000; Synaptic Systems), CDX2 (1:500; Biogenex), Pd×1 (1:5000; Abcam; data not shown). All secondary antibodies (AlexaFluor; Invitrogen) are used at 1:500 dilution. Confocal microscopy images are captured with a 20× plan apo objective on a Nikon A1Rsi Inverted, using settings of 0.5 pixel dwell time, 1024 resolution, 2× line averaging, and 2.0× A1 plus scan.

Total RNA is extracted from HIOs using a NucleoSpin RNA II kit (Macherey-Nagel), and cDNA is synthesized with SuperScript VILO (Invitrogen) using 300 ng RNA. qPCR analysis is performed with TaqMan Fast Advanced Master Mix and custom designed TaqMan Array 96-Well FAST Plates (Applied Biosystems) consisting of the following targets: 18S—Hs99999901_s1; GAPDH—Hs999999905_m1; ARX—Hs00292465_m1; CHGA—Hs00900370_m1; SYP—Hs00300531_m1; NTS—Hs00175048_m1.

Clinical Phenotypes of Congenital Diarrhea Disorders:

Congenital diarrhea disorders comprise a heterogeneous group of diseases composed of rare enteropathies related to specific etiology and pathogenesis including: (i) defects in absorption and transport of nutrients and electrolytes; (ii) maintenance and differentiation of enterocytes; (iii) differentiation and function of enteroendocrine cells (EECs) and (iv) modulation of the intestinal immune response⁷. This potentially life threatening condition in young infants and children is defined as congenital, severe, non-infectious diarrhea lasting more than two weeks, with consequent malabsorption, multiple food intolerance and failure to thrive^{5,6}. Since this condition cannot be successfully treated, affected individuals depend on life-long Parenteral Nutrition (PN) and in some cases small bowl transplantation⁸.

Origins and Relationships of Patients:

Eight patients from seven different families of Jewish Iraqi origin with an apparent autosomal recessive pattern of malabsorptive diarrhea, originally defined as having intractable diarrhea of infancy syndrome (IDIS)⁷ are studied. Identity By Descent (IBD) analysis confirm the family relations and indicated that the closest cross-family relationship had IBD=0.040.

Mapping of Deletions in Patients:

Exome sequencing analysis of 5 patients (FIG. 4) reveal no rare exonic sequence variants with the appropriate patient segregation. Whole genome linkage analysis (FIG. 5) and haplotype reconstruction using SNP genotyping is performed on 6 of the patients in families 1-5 and their 22 relatives detected a single significant (LOD score=4.26) telomeric linkage interval on chr16 with flanking marker rs2074359 (chr16:2,984,868). Recombination analysis using both SNP genotyping and exome data (when available) reduce the linkage interval to a 800 kb region within the linkage interval on chr16: 1,050,877-1,849,916, in the 4 patients of families 1, 2, 3, 5. To explore possible genomic structural variations, exome sequence read coverage is examined in the interval and discovered zero coverage of the first 3 exons of a predicted transcript of C16ORF91, suggesting a homozygous copy number variation (CNV) deletion. PCR amplification and Sanger sequencing in these families revealed a 7,013 bp deletion (FIG. 1A to 1G). Further scrutiny by database searches and quantitative RT-PCR showed that these three exons are non-transcribed, i.e. mistakenly included in the exome capture kit, suggesting that the ΔL region is intergenic. Two patients in family 4, who did not share the region of homozygosity, are found to be compound heterozygotes for ΔL along with a distinct allelic variant ΔS, a partially overlapping 3101 bp deletion (FIG. 1A to 1G). Families 6 and 7 respectively showed the ΔS/ΔS and ΔL/ΔL genotypes (FIG. 1A to 1G). A 1,528 bp region, termed ICR, is defined as the overlap of ΔL and ΔS FIG. 1A to 1G), is inferred to be a disease critical region, as it is homozygously deleted in all affected individuals. Patient 1.1 showed uniparental isodisomy for the maternal chromosome carrying the ΔL allele.

Whole Genome Sequencing Controls:

Whole-genome sequencing for patient 2.1 confirmed the ΔL attributes and showed that it is the only homozygous genomic deletion in the linked region. None of the deletions are present in 200 ethnically matched Iraqi control chromosomes as well as in either 122 in-house Caucasians WGS samples. In addition, >3000 WGS of diverse sources in the KAVIAR dataset⁴² are searched and no deletions overlapping are found. Further, 1092 individuals from the 1000 Genome Project⁴³ are scanned within the integrated variant calls file (ALL.wgs.integrated_phasel_v3.20101123.snps_indels_sv.sites.vcf), seeking overlaps with the ⊗L and ⊗S regions, and no such are observed. Searching the Database of Genomic Variants^{44,45} for large deletions that span the ⊗L and ⊗S regions identified several heterozygous deletions with combined allele frequency <0.004.

Mouse Microbiome Dysbiosis:

The fecal samples of knockout mice exhibit considerably reduced microbial diversity with respect to WT feces (FIGS. 8 and 9A to 9C). This loss of microbial diversity is indicated both by significantly fewer unique microbial OTUs in knockout vs WT feces samples, as well as the overabundance of just a few bacterial genera in the knockout that are not typically enriched in the WT samples (FIG. 9A to 9C).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A recombinant or isolated polypeptide comprising at least 70% identity of SEQ ID NO: 1, SEQ ID NO:2, or SEQ ID NO:3.

2. The recombinant or isolated polypeptide of claim 1, wherein the polypeptide comprises one or more of the following amino acid sequences: MAAGVIR (SEQ ID NO: 4), SEEEEEEEEEEEEEE (SEQ ID NO: 5), SPETP (SEQ ID NO: 6), QLLRFSELIS (SEQ ID NO: 7), RYFGRKD (SEQ ID NO: 8), GQDPDA (SEQ ID NO: 9), LYYADLV (SEQ ID NO: 10), PLGPLAELFDYGL (SEQ ID NO: 11), LERKY (SEQ ID NO: 12), HITPM (SEQ ID NO: 13), QRKLPPSFWKEP (SEQ ID NO: 14), PLGLLH (SEQ ID NO: 15), and GTPDFSDLLASWS (SEQ ID NO: 16).

3. A nucleic acid encoding the polypeptide of claim 1.

4. A host cell comprising the nucleic acid of claim 3 capable of expressing the polypeptide.

5. A transgenic non-human mammal, wherein the mammal is deleted or knocked out for one or more of an intestine-critical region (ICR).

6. A pharmaceutical composition comprising the polypeptide of claim 1 and a pharmaceutically acceptable carrier.

7. A method of treating or preventing a subject suffering or at risk or suspected of suffering from a diarrhea disease or disorder, the method comprising administrating a pharmaceutical composition of claim 6 to a subject in need of such treatment.


SEQUENCE LISTING (47 sequences)

SEQ ID NO: 1 - protein, 272 aa, Mus musculus
MAAGVIRSVCDFRLPLPSHESFLPIDLEAPEISEEEEEEEEEEEEEEEEEEVDQDQQGEGSQGCGPDSQSSGVVPQDPSSPETPMQLLRFSELISGDIQRYFGRKDTGQDPDAQDIYADSQPASCSARDLYYADLVCLAQDGPPEDEEAAEFRMHLPGGPEGQVHRLGHRGDRVPPLGPLAELFDYGLRQFSRPRISACRRLRLERKYSHITPMTQRKLPPSFWKEPVPNPLGLLHVGTPDFSDLLASWSAEGGSELQSGGTQGLEGTQLAE

SEQ ID NO: 2 - protein, 266 aa, Homo sapiens
MAAGVIRPLCDFQLPLLRHHPFLPSDPEPPETSEEEEEEEEEEEEEEGEGEGLGGCGRILPSSGRAEATEEAAPEGPGSPETPLQLLRFSELISDDIRRYFGRKDKGQDPDACDVYADSRPPRSTARELYYADLVRLARGGSLEDEDTPEPRVPQGQVCRPGLSGDRAQPLGPLAELFDYGLQQYWGSRAAAGWSLTLERKYGHITPMAQRKLPPSFWKEPTPSPLGLLHPGTPDFSDLLASWSTEACPELPGRGTPALEGARPAE

SEQ ID NO: 3 - protein, 300 aa, Mus musculus
MHVEPLLHPSACVCCSREPQNFGDLNKMAAGVIRSVCDFRLPLPSHESFLPIDLEAPEISEEEEEEEEEEEEEEEEEEVDQDQQGEGSQGCGPDSQSSGVVPQDPSSPETPMQLLRFSELISGDIQRYFGRKDTGQDPDAQDIYADSQPASCSARDLYYADLVCLAQDGPPEDEEAAEFRMHLPGGPEGQVHRLGHRGDRVPPLGPLAELFDYGLRQFSRPRISACRRLRLERKYSHITPMTQRKLPPSFWKEPVPNPLGLLHVGTPDFSDLLASWSAEGGSELQSGGTQGLEGTQLAEV

SEQ ID NO: 4 - protein, 7 aa, Mus musculus
MAAGVIR

SEQ ID NO: 5 - protein, 15 aa, Mus musculus
SEEEEEEEEEEEEEE

SEQ ID NO: 6 - protein, 5 aa, Mus musculus
SPETP

SEQ ID NO: 7 - protein, 10 aa, Mus musculus
QLLRFSELIS

SEQ ID NO: 8 - protein, 7 aa, Mus musculus
RYFGRKD

SEQ ID NO: 9 - protein, 6 aa, Mus musculus
GQDPDA

SEQ ID NO: 10 - protein, 7 aa, Mus musculus
LYYADLV

SEQ ID NO: 11 - protein, 13 aa, Mus musculus
PLGPLAELFDYGL

SEQ ID NO: 12 - protein, 5 aa, Mus musculus
LERKY

SEQ ID NO: 13 - protein, 5 aa, Mus musculus
HITPM

SEQ ID NO: 14 - protein, 12 aa, Mus musculus
QRKLPPSFWKEP

SEQ ID NO: 15 - protein, 6 aa, Mus musculus
PLGLLH

SEQ ID NO: 16 - protein, 13 aa, Mus musculus
GTPDFSDLLASWS

SEQ ID NO: 17 - DNA, 20 bp, Artificial Sequence
  Primer for determining deletion boundary
catgtgccgcatctctggac

SEQ ID NO: 18 - DNA, 20 bp, Artificial Sequence
  Primer for determining deletion boundary
ggaccgtggagtgtttgtgc

SEQ ID NO: 19 - DNA, 22 bp, Artificial Sequence
  Primer for determining deletion boundary
cagtggagatggtcatggctgt

SEQ ID NO: 20 - DNA, 21 bp, Artificial Sequence
  Primer for determining deletion boundary
tcttcctcctccgaagtctct

SEQ ID NO: 21 - DNA, 22 bp, Artificial Sequence
  Primer for determining deletion boundary
aaacaggtgcctctgttgacac

SEQ ID NO: 22 - DNA, 24 bp, Artificial Sequence
  Primer for determining deletion boundary
caatctcaactcactgcaacctct

SEQ ID NO: 23 - DNA, 22 bp, Artificial Sequence
  Primer for polymorphic marker
gcccggtcataaattgttgtat

SEQ ID NO: 24 - DNA, 21 bp, Artificial Sequence
  Primer for polymorphic marker
tctgccaaaagtctaggtgtg

SEQ ID NO: 25 - DNA, 20 bp, Artificial Sequence
  Primer for polymorphic marker
gcctgtggatggtgaatttt

SEQ ID NO: 26 - DNA, 20 bp, Artificial Sequence
  Primer for polymorphic marker
actacaggtgccaccaccac

SEQ ID NO: 27 - DNA, 18 bp, Artificial Sequence
  Primer for polymorphic marker
cacgctcgcactcgtatg

SEQ ID NO: 28 - DNA, 20 bp, Artificial Sequence
  cctgacgctcagctaggaag
cctgacgctcagctaggaag

SEQ ID NO: 29 - DNA, 21 bp, Artificial Sequence
  Primer for polymorphic marker
atggcccctgtatgtcttttc

SEQ ID NO: 30 - DNA, 20 bp, Artificial Sequence
  Primer for polymorphic marker
aaacaacagctgggcatggt

SEQ ID NO: 31 - DNA, 22 bp, Artificial Sequence
  Primer for polymorphic marker
atcctcgtgctatgaacagaca

SEQ ID NO: 32 - DNA, 22 bp, Artificial Sequence
  Primer for polymorphic marker
gagcactattctgcctcccata

SEQ ID NO: 33 - DNA, 22 bp, Artificial Sequence
  Primer for polymorphic marker
ccatagtttctaaccctcagca

SEQ ID NO: 34 - DNA, 22 bp, Artificial Sequence
  Primer for polymorphic marker
atggaatgttagcattggctct

SEQ ID NO: 35 - DNA, 19 bp, Artificial Sequence
  Primer for polymorphic marker
tgatgagctctgaaaagcg

SEQ ID NO: 36 - DNA, 19 bp, Artificial Sequence
  Primer for polymorphic marker
gaacctgcccctctgtctc

SEQ ID NO: 37 - DNA, 29 bp, Artificial Sequence
  Primer for generating or assessing ICR deletion
atccagcacaccctcagctttaactagtc

SEQ ID NO: 38 - DNA, 30 bp, Artificial Sequence
  Primer for generating or assessing ICR deletion
cattctttggtcacatacaggtgggacctt

SEQ ID NO: 39 - DNA, 26 bp, Artificial Sequence
  Primer for generating or assessing ICR deletion
aggtatggtgggagatggggtagtca

SEQ ID NO: 40 - DNA, 28 bp, Artificial Sequence
  Primer for generating or assessing ICR deletion
agccatgtctaggctccaaagtgagaac

SEQ ID NO: 41 - DNA, 26 bp, Artificial Sequence
  Primer for generating or assessing ICR deletion
ttggctggacgtaaactcctcttcag

SEQ ID NO: 42 - DNA, 26 bp, Artificial Sequence
  Primer for generating or assessing ICR deletion
ctagtcctcacacccagctctttcaa

SEQ ID NO: 43 - DNA, 30 bp, Artificial Sequence
  Primer for generating or assessing ICR deletion
cctagaacttgctatataaactggacaagc

SEQ ID NO: 44 - DNA, 30 bp, Artificial Sequence
  Primer for generating or assessing ICR deletion
gtgaagcgctggacggagagataatcagta

SEQ ID NO: 45 - DNA, 26 bp, Artificial Sequence
  Primer for generating or assessing ICR deletion
gtgtcttctctgtcctcctggagtca

SEQ ID NO: 46 - DNA, 28 bp, Artificial Sequence
  Primer for generating or assessing ICR deletion
gttctcactttggagcctagacatggct

SEQ ID NO: 47 - DNA, 29 bp, Artificial Sequence
  Primer for generating or assessing ICR deletion
gactagttaaagctgagggtgtgctggat